(19) United States
(12) Patent Application Publication
(10) Pub. No.: US 2004/0033589 A1
(43) Pub. Date: Feb. 19, 2004

(54) Biolistic device

(76) Inventor: John Anthony O'Brien, Cambridge (GB)

Correspondence Address: William M Lee Jr, Barnes & Thornburg, PO Box 2786, Chicago, IL, 60690-2786, US

(21) Appl. No.: 10/380,452

(22) PCT Filed: Oct. 12, 2001
(86) PCT No.: PCT/GB01/04564

(30) Foreign Application Priority Data
Oct. 20, 2000 (GB) 0025710.5

(51) Int. Cl. C12M 1/00

(52) U.S. Cl. 435/285.3

(57) ABSTRACT

A biolistic device for accelerating a carrier gas bearing particles is provided. The device comprises an elongate body (36) with a cylindrical channel (40) extending therethrough, wherein a first end of the channel is adapted to receive gas for acceleration and a second end of the channel is adapted to direct gas to a target. A less dispersive output beam is produced which increases the depth and penetration of the beam for a given gas pressure and provides transfection of cells over a greater depth. The elongate body (60) comprises an accelerating section (80, 82), and an output section (84) the channel (64) extending through both the accelerating section and the output section, wherein the output section has a plurality of apertures extending at an angle from an inner wall of the output section to an outer wall of the output section. These apertures (88) reduce gas turbulence within the output section and result in a laminar flow of carrier gas from the output orifice.

### FIELD OF THE INVENTION

This invention relates to a biolistic device for transfecting genetic material.

### BACKGROUND TO THE INVENTION

Biolistic delivery technology was first introduced (Klein et al., 1987 & 1988) as a method of gene transfer into monocotyledonous plants. Biolistics is a method where small particles coated with DNA are fired into cells by rapid propulsion in a carrier gas, usually helium. The BIO-RAD gene gun is a hand-held biolistic device that provides a physical method of transfecting cells with DNA and is not dependent on specific ligand-receptors or any biochemical features. However the BIO-RAD gene gun produces a beam of limited penetration which is generally only suitable for in-vitro modification of certain cells.

It is the aim of the present invention to provide a biolistics device that is suitable for in-vivo use and capable of modifying the genetic structure of cells that have proved resistant to modification by existing biolistic devices

### SUMMARY OF THE INVENTION

In accordance with one aspect of the present invention, there is provided a biolistic device for accelerating a carrier gas bearing particles, the device comprising an elongate body with a cylindrical channel extending therethrough, wherein a first end of the channel is adapted to receive gas for acceleration and a second end of the channel is adapted to direct gas to a target, thereby to produce a compact output beam of carrier gas from the output orifice. The substantially constant cross-section and smooth surface of the channel reduces turbulence imparted to the carrier gas as it is accelerated along the channel, so substantially reducing the coanda effect seen in prior art devices. A less dispersive output beam is thus produced which increases the depth and penetration of the beam for a given gas pressure. By having a beam which is more focussed, transfection occurs over a greater depth and damage to cells is substantially reduced.

Preferably the elongate body comprises an accelerating section, and an output section, the channel extending through both the accelerating section and the output section, wherein the output section has a plurality of apertures extending at an angle from an inner wall of the output section to an outer wall of the output section. These apertures reduce gas turbulence within the output section, in part due to a portion of the carrier gas being allowed to leave the output section via these apertures, and result in a laminar flow of carrier gas from the output orifice.

The apertures are preferably inclined at an angle of 30°, with typically twenty apertures being provided, five groups of apertures being regularly spaced about the circumference of the outer section, each group consisting of four apertures spaced apart along the length of the output section.

A mesh is preferably disposed over the output orifice so as to reduce agglomeration of particles in the output beam of carrier gas. Typically the mesh is provided by 70 μm nylon mesh, although mesh made from any type of plastics material may be used. The mesh may be secured over the output orifice by an annular member, such as a metal ring.

The cylinder is preferably made of plastics material, such as Delrin, with the output section being made either of plastics material or of metal, such as brass.

Where the elongate body comprises a separable accelerating section and output section, the size of the channel may remain constant along the length of the body or may vary in size.

Thus the internal diameter of a portion of channel within the accelerating section may be 2.9 mm with the internal diameter of a portion of channel within the output section 4.5 mm.

To allow easy alteration of the channel size, the accelerating section is preferably provided with a cylindrical recess, the recess engaging with the output section with a pressfit connection. The accelerating section may then be used with output sections having different internal diameters, allowing the characteristics of the output beam to be altered. Thus the device may be supplied with a number of different output sections having a common outer diameter, and each having a different constant internal diameter.

Preferably the elongate body is substantially T-shaped, with sealing means provided at the base of the T, and a connecting means provided on the neck of-the T, thereby to allow the body to be retrofitted into existing biolistic devices. Typically the sealing means will be provided by at least one o-ring, with the connecting means being a screw-thread capable of mating engagement with a corresponding threaded portion within a biolistic device.

A guide rod may be attached to set the distance of the device from a target.

Thus in accordance with another aspect of the invention, there is provided an elongate body with a central channel having a smooth inner wall for retrofitting to a BIO-RAD gene gun. The elongate body preferably comprises the preferred features discussed above.

The invention also lies in a biolistic device as aforesaid when used for iii-vivo transfection.

The invention will now be described, by way of example, with reference to the accompanying drawings in which:

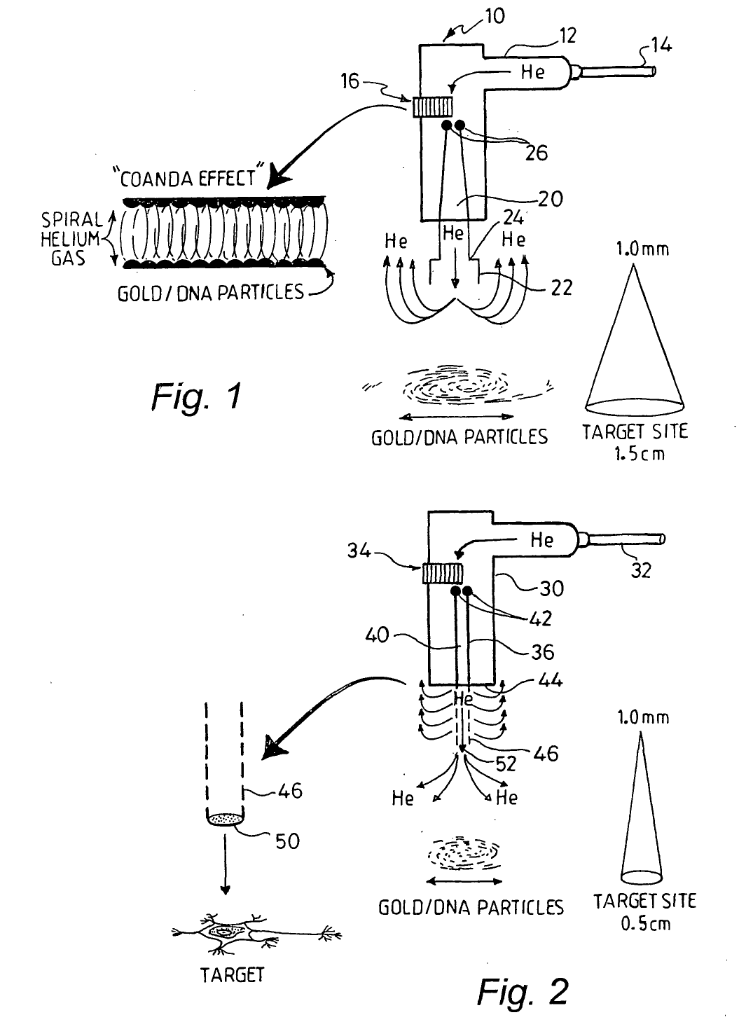

FIG. 1 shows a schematic diagram of a prior art gene gun;

FIG. 2 shows a schematic diagram of a gene gun in accordance with the present invention;

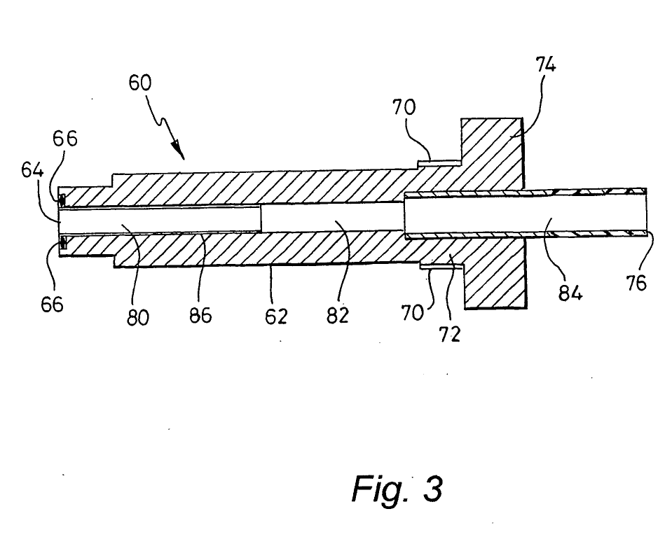

FIG. 3 shows a body for retrofitting to an existing gene gun;

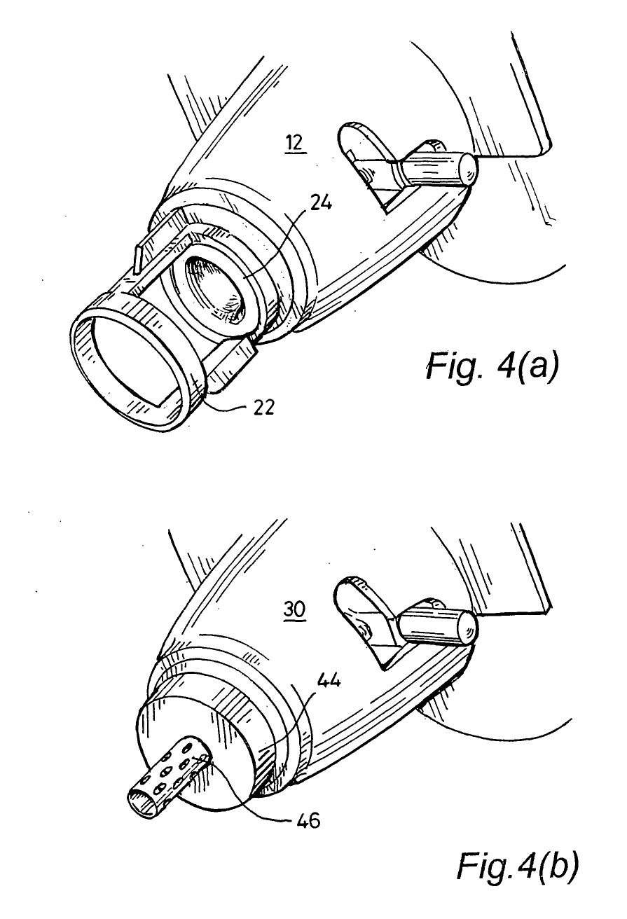

FIG. 4 shows a perspective view from the front of a prior art gene gun and a gene gun in accordance with the present invention;

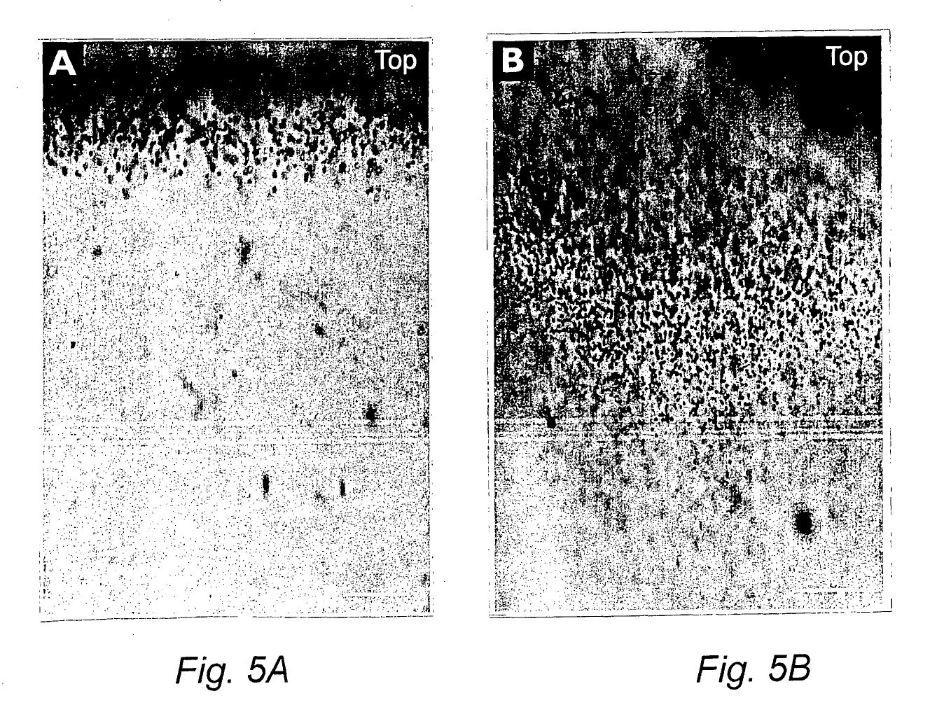

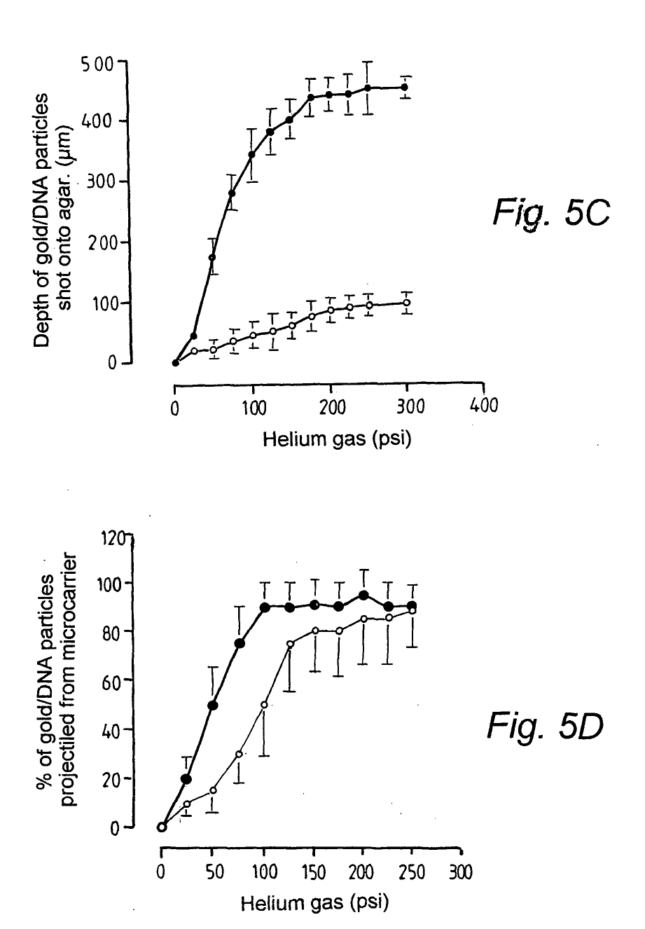

FIG. 5 shows a comparison of the effects of the gene guns shown in FIGS. 1 and 2; and

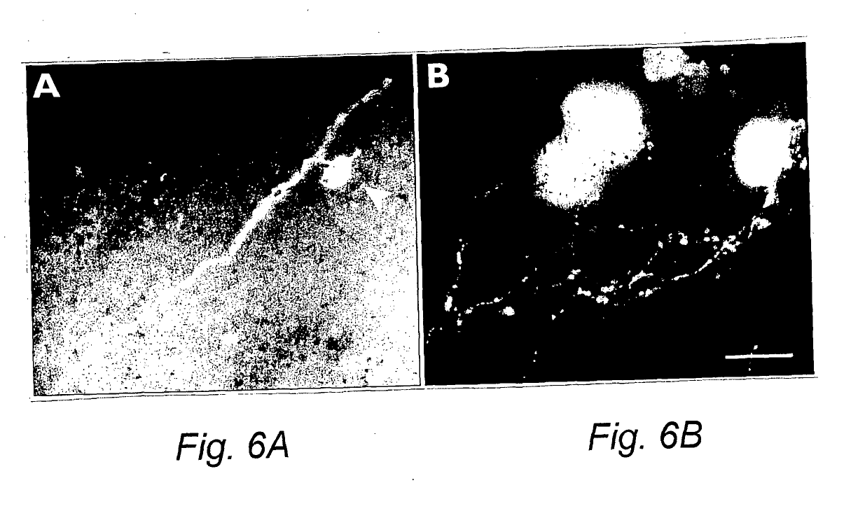

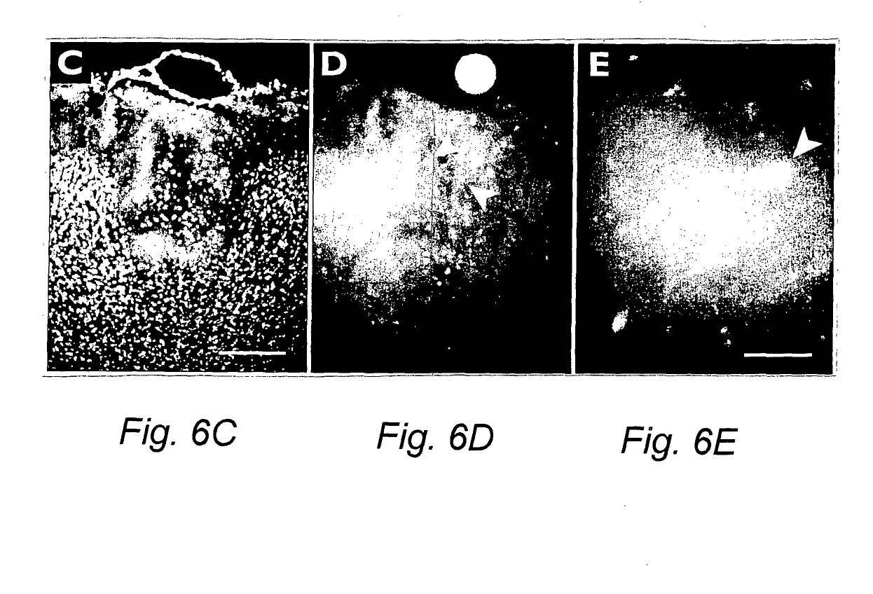

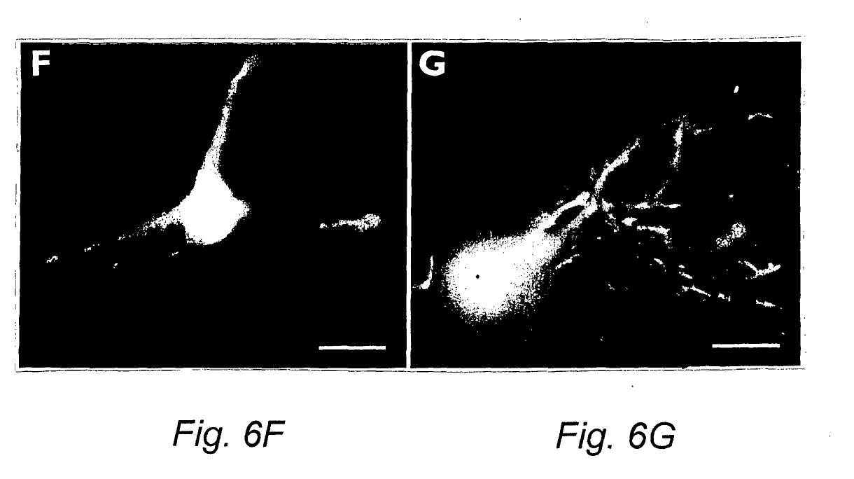

FIG. 6 shows examples of biolistically transfected cells achieved using the gene gun in accordance with the present invention.

### DESCRIPTION

FIG. 1 shows a known gene gun 10 as sold by Bio-Rad under Catalogue Numbers 165-2431 and 165-2432, the operation and structure of which is fully described in the Helios Gene Gun System Instruction Manual from Bio-Rad and which is herein incorporated by reference. The gun 10 comprises a body 12 with an inlet 14 for helium gas, a cartridge holder 16, a barrel 20 and a spacer 22 at an output end 24 of the barrel 20. The barrel 20 has o-rings 26 at its upper end to ensure a seal with the helium supply and diverges towards an outlet of the gun, so as to be substantially cone shaped. Gold/DNA particles, adhered to the surface of Tetzel tubing, are placed into the gene gun cartridge holder 16. When a certain amount of pressure of helium (above threshold) is fired into the gun, the DNA/gold particles are released from the plastic microcarrier, or cartridge, and become entrained in the helium stream. The velocity of helium gas must be sufficient to allow penetration of gold/DNA particles into the cell membranes to transform cells. The BIO-RAD gene gun, in principle, focuses on the ‘coanda phemomena’ increasing the spread of particles over a given target via helium (Imants R 1966). Thus, when a pulse of helium is fired into the gene gun, the helium rapidly expands several fold as it travels down the cone-shaped barrel. The expanding gas causes the gold/DNA particles to spiral down the accelerating channel with an increasing velocity. As soon as the helium leaves the cone barrel it will follow the contours of the outer surface, this allows the gold/DNA particles to accelerate out of the barrel liner in a continuing outward spiral motion and creating a dispersed divergent beam at the outlet. The particles retain velocity until they emerge from the channel and hit a target. Typically the beam will cover a target circular site of 1.5 cm diameter when a target is placed adjacent to the spacer.

Both stable and transient expressions can be introduced into cells using this particle bombardment and no extraneous genes or proteins are delivered. The system can transfect a small number of cells and only a small amount of DNA is required. The most important factor in achieving successful transfections through biolistics is the biological state of the tissue following bombardment. Survival of bombarded cells will depend on their initial viability; tissue must remain healthy and must retain the potency to express new genes. The number of cells expressing the transfected DNA plasmid is directly proportional to cell survival (Wellmann et al., 1999). There are other biological parameters that are important for successful biolistic transfection. First, one must have an appropriate gene construct with a promoter that will express in the desired target tissue. Second, the target cells must be in a state receptive to transformation and third, there must be a high rate of particle penetration within the tissue.

Choice of particle is an important parameter. There are currently two sorts of microprojectiles which have been successfully applied in biolistic DNA-transfer experiments; tungsten and gold particles. Gold is used in preference to tungsten because gold particles are non-toxic to neurons, uniform in shape and size and the DNA coated particles can be stored for weeks at 4° C. in a desiccated environment. Tungsten particles acidify culture medium, inducing apoptosis of neurons. They are also irregular in shape and heterogeneous in size. In addition, the DNA on tungsten particles is more susceptible to catalytic degradation than DNA on gold particles (Sanford et al., 1993).

Helium is the propellant of choice because it is biologically inert and highly diffusible. Any gas at sufficient pressure will cause a shock wave, which spreads ahead of the expanding gas. This shock wave can cause a ‘deadzone’ of cells. Cell death is not caused by the DNA-coated particles themselves but by the blast of helium accompanying them. To minimise tissue damage, an optimal distance between the barrel of the gene gun and the target cells must be determined. This should take into account a decreased likelihood that a gold/DNA particle will penetrate the target if the distance from the barrel to the tissue is too great (Wellmann et al., 1999).

The hand-held gene gun has been extensively used on epidermal cells of the skin of vertebrates (Yang et al., 1990). In such in-vivo experimental systems the gene gun was applied to the skin for vaccination with limited success. The transfection efficiency in skin is approximately 10-20% (Williams et al., 1991). While transfection of skin is reproducible and yields high levels of transgene expression, the majority of the transfected cells in the epidermis were keratinocytes (Andree et al., 1994), and in the dermis most are in the panniculus carnosus layer (Cheng et al., 1993). Bombardment of internal soft tissue has been more difficult. Large differences in transfection efficiency between tissues have been reported. Rat epidermis showed 1000 times more pCMVluc expression compared to muscle tissue (Cheng et al., 1993, Hui et al., 1994, Yang et al., 1996). It has also been demonstrated that in mice muscle gold/DNA particles penetrate only the outer layer while in the same study several layers of spleen were transfected (Hui et al., 1994). Thus cell types are distinct in their sensitivity to particle bombardment. In addition, among different bombardment tissues the expression level of a reporter gene in time differs as well as the potency.

Transfection using lipofection and calcium phosphate precipitation depends on cellular uptake. Non-dividing, fully differentiated neuronal cells are often not successfully transfected using the above methods (Jiao et al., 1992). Modifications of the lipofection protocol using polycations have improved efficiencies but there are still physical limitations in cellular processing (Dong et al., 1993). Electroporation is less dependent on cellular uptake but it is a highly destructive technique with a cell viability dropping to 5-50% (Oellig et al., 1990). Viral vectors effectively transfect neurons both in-vitro and in-vivo but the method is labour-intensive, difficult to control, and often cell viability is compromised (Katz et al., 1994).

Neurons are the most difficult cells to transfect due to their non-dividing, fully differentiated nature and the surrounding glial cell providing protection (Biewenga et al., 1997). Since differentiated neuronal cells are postmitotic the transport of plasmid DNA to the nucleus may be inefficient and probably contributes to their resistance to traditional transfection techniques (Katz et al., 1994). Particle-mediated gene transfer into live neuronal tissue is also difficult due to the fragile nature of the tissue (Sato et al., 2000). No technique of transfection and subsequent expression of DNA in living neuronal tissue has yet emerged that is non-damaging, efficient and reproducible.

The BIO-RAD gene gun shown in FIG. 1 delivers particles superficially over a relatively wide area ideal for cultured cells (monolayers). Organotypic cerebellum slices reveal that this BIO-RAD gun gives a gold/DNA particle penetration depth less than 100 μm (data not shown). This is because the cone-shaped bore of the barrel 20 causes the gold/DNA particles to spread outwards from their original 1 mm diameter, resulting from the cartridge holder, to a diameter of 1.5 cm at the target site. Further agglomeration of the particles occurs, producing clumps of particles that travel further, faster and cause more damage than single particles. For these reasons accurate use of the BIO-RAD gene gun for transfecting of deep tissues is not possible.

A modified gene gun in accordance with the present invention is shown in FIG. 2.

When using this gun, firstly DNA coated particles were prepared for placing in the gun. An encoding yellow fluorescent protein (EYPF) plasmid DNA (50 μg) is attached to 25 mgs of gold particles having a 1 μm diameter by precipitation with polycation spermidine (50 μl of 0.05M) and CaCl₂ (50 μl of 10 mM). The gold/DNA slurry is extensively washed with absolute ethanol and is resuspended in ethanol with polyvinylpyrrolidine (PVP). The optimum amount of PVP is in the range 0.05 to 0.1 mg/ml with the preferred concentration being 0.075 mg/ml. The DNA slurry is coated onto the inner wall of tubing (Tetzel tube; BIO-RAD Laboratories) and the DNA particles allowed to settle for a brief time in the Tetzel tube before the supernatant is removed. The Tetzel tube is rotated at 180° to achieve an even film of gold/DNA and a slight flow of nitrogen is used to evaporate the remaining ethanol. The tubing is cut into 50 mm length cartridges and these are inserted into a cartridge holder in the modified gene gun ready to be fired, and stored at 4° C.

The gun shown in FIG. 2 comprises a body 30 with an inlet 32 for helium gas and a cartridge holder 34. However instead of using a cone-shaped barrel as for the prior art gene gun shown in FIG. 1, acceleration and travel of gas occurs along an elongate hollow cylinder 36 of substantially constant diameter, having a smooth inner wall. The diameter of a channel 40 surrounded by the cylinder is approximately 5 mm, as compared to a diameter of 15 mm for the gun in FIG. 1. O-rings 42 are used to seal an upper end of the cylinder and ensure that all helium supplied to the gun enters the channel. At an end face 44 of the body, the channel extends into a barrel 46 of length 16 mm. A nylon mesh 50 of 70 μm particle size is placed over the end of the barrel 46 so as to reduce particle agglomeration in the output beam 52 of helium. The beam spread produced on operation of the beam is 0.5 cm, as compared to a spread of 1.5 cm on the BIO-RAD gene gun for a target at the same distance.

A detailed view of a body which can be inserted into an existing gene gun to modify it in accordance with the present invention is shown in FIG. 3, and this body has the same features as cylinder 36 shown in FIG. 2. The body 60 comprises a T-shaped plastics section 62, made of Delrin, with a central cylindrical bore 64. O-rings 66 are provided at one end, and a screw-threaded portion 70 provided at neck 72. The O-rings 66 and screw-threaded portion 70 allow the body 60 to retrofit into an existing gun to modify it to act in accordance with the present invention. The hollow bore 64 can be the same diameter along its length and extend beyond T-piece 74 into a barrel 76 of the same internal diameter.

Alternatively as shown in FIG. 3, the bore 64 can vary slightly in cross-section along its length. Thus in FIG. 3 the bore 64 comprises an inner 80, a middle 82 and an outer section 84, the inner section 80 comprising an hypodermic tube 86 of outer diameter 3.75 mm and inner diameter 2.9 mm press-fitted into the innermost end of the bore, and the middle section 82 being defined by walls of Delrin. The outer section 84 comprises barrel 76, made of brass tube of outer diameter 6.3 mm and inner diameter 4.5 mm, which is received in the bore due to an increase in diameter in the bore at one end, the tube extending beyond the bore by a distance of 16 mm. The barrel 76, where it protrudes beyond the bore, has inclined baffle holes 88 drilled into it an angle of 30°, a group of four baffle holes being spaced apart along the length of exposed section of barrel 76, and repeated around the circumference at five equidistant points, creating a total of 20 holes.

A brass ring 90 fits securely over the end of the exposed section of barrel 76 and is used to hold nylon membranes or meshes 92 of between 5 micron-100 microns spacing at the end of the barrel. The membrane 92 prevents particle agglomerations created in the barrel from impacting on the sample and creating tissue damage in the sample as the agglomerations are either retained in the barrel by the mesh or broken up by the mesh before reaching the sample. Thus the use of the brass ring allows meshes of variable size to be interchanged easily as necessary.

A guide rod 94 made of stainless steel screws into a co-operating recess at the end of body 62 and is used to fix the exact distance between the gun and sample. Rods of varying length can be screwed into the recess, and typically the rods range in length from 6 cm to 3 cm. Thus in use, the gun is placed directly above the sample so that the end of the guide rod touches the sample and so fixes the distance between sample and gun accurately without the random variation in distance that would occur if the gun was merely held above the target without any guide to distance.

Detailed views of a prior art gun and the modified gun are shown in FIGS. 4(a) and 4(b), showing the spacer 22, and apertured barrel 46.

With the gun in accordance with the present invention, increased gold/DNA particle depth penetration is achieved without damaging tissue. Transfection of a wider range of cells is thus possible. By altering the design of the channel through which the carrier gas and particles travel, lower gas pressures can be used to achieve increased penetration.

Where appropriate, particle size and gas velocity can also be altered. By forming a more compact group of particles, a greater particle penetration can be achieved for a smaller volume of tissue.

The gene gun in accordance with the present invention has a reduced “spread” of particles at its output and produces a beam with increased penetration within tissue. The design of the channel allows significant penetration for reduced gas pressure, so allowing a reduction in the helium shock wave which can cause tissue damage (Sanford et al., 1993). The mesh also deflects the gas blast away from the target cells further reducing the shock wave and distributing the particles more uniformly.

The use of a bore with a substantially constant or similar cross section reduces the Coanda effect on helium, thus preventing an outward spiral formation of gold/DNA particles, so reducing spread on target site. Schematic representations of the spread seen for respective guns are shown in FIGS. 1 and 2.

Comparison of the properties of the BIO-RAD gene gun and a gun in accordance with the present invention is now presented.

To investigate the optimum conditions for gold/DNA particles penetrating neurons using the gene gun of FIG. 2, 5% agar plates were used. Gold/DNA particles were fired onto agar plates at gas pressures ranging from 25 to 300 psi. Penetrations of particles onto agar were measured using a Radiance confocal microscope (2000 BIO-RAD) with X40 objective lens.

Considering the BIO-RAD gun, a non-linear relationship was found between pressure and depth with penetration reaching a maximum of 100 μm with an optimum gas pressure of 150 psi, as shown at FIG. 5A where calibration scale bars are 50 μm. The gun of FIG. 2 achieved penetration of approximately 275 μm for gas pressure of 75 psi, see FIG. 5B where calibration scale bars are 50 μm. When helium gas pressure was set at 200 psi in the BIO-RAD gun, a penetration value of approximately 100 μm was achieved, whilst the gun of FIG. 2 reached values greater than 400 μm for 200 psi. Thus, with a helium gas pressure of 200 psi we observed a significant 4-fold depth increase in penetration over the BIO-RAD gun, see FIG. 5C, where open circles represent the BIO-RAD gun and closed circles the gun of FIG. 2. However, beyond 200 psi there was no difference in performance between the BIO-RAD gun and the gun in accordance with the present invention; increasing the gas pressure does not further increase the penetration.

Reducing the gas pressure from 150 psi to 75 psi in the gun of FIG. 2 still effectively removed most of the gold/DNA particles inside the microcarrier, see FIG. 5D where the efficiency of each microcarrier was checked to assess how much gold/DNA was left on them after they were shot (once only) at a given pressure. Below 50 psi the particles were not effectively removed from the microcarrier. A desirable penetration made by the modified BIO-RAD gene gun, must have a gas pressure greater than 50 psi. Thus, a gas pressure of 75 psi gives us an appropriate penetration depth of 275 μm with 80% gold/DNA removal efficiency from a microcarrier.

Transfection of different types of tissue was carried out using the two types of gun.

Neuronal Transfection

Explants and organotypic slices were bombarded with 1.0 μm gold particles coated with CMV(cytomegalovirus)-driven EYPF expression plasmid, which provides excellent labelling, with a filter block set (XF105 with a long pass emitter, Glen Spectra Ltd) that reduces autofluorescence, giving the best possible signal-to-noise ratio. A microcarrier loading quantity (MLQ) of 1.0 μm gold particle size and 2 μg DNA/mg were found to provide optimum transfection efficiency for both tissue types. We observed detectable tissue damage when organotypic slices or explants was subjected to 1.6 μm gold particle bombardment and 2 μg DNA/mg (data not shown) using the gun of FIG. 2. No detectable transfection was observed for the BIO-RAD gun.

Transfection of Fish Organotypic Slices

Fresh elephant nose fish brains were removed and cut on a McIlwain tissue chopper (Manufactured by Mickle Laboratory Engineering Co Ltd) at either 300 μm or 400 μm. The slices of cerebellum were placed on a membrane support in six well sterile tissue culture plates (0.4 μm Millicell-CM, Millipore). Culture media was placed underneath the membrane (Katz et al., 1994) thus the upper surface of the slice was exposed to the incubator atmosphere while the lower surface contacted culture medium. Every second day the medium was replaced (fish culture medium comprises of DMEM (Dulbecto's Modified Eagle's Medium) & Medium 199 (1:1), 1 mM Hepes, 1% Essential Amino Acids, Penicillin-Streptomycin 100 iu/ml). Between four and eighteen days in culture the organotypic slices were ‘shot’ with EYPF CMV-driven expression plasmids (Clontech) and incubated for a further ten to fourteen days at 22° C. 5% Co₂. Fish cerebellum cells expressing EYFP were detected after five days and expression intensity peaked fourteen days following biolistic transfection. All slices were fixed in 4% Paraformaldehyde (PFA) for 20 min at room temperature, washed in phosphate buffered saline (PBS) followed by counterstaining in 0.01% Hoechst 33342 for 2 min. Slices were mounted using vectashield and analysed by confocal microscopy.

Successful neuronal transfection was observed in both gene gun designs, and was easily identified by characteristic dendritic arbours, spines and axonal trajectories, see FIG. 6F where transfected neurones were identified at 100 μm within the organotypic slice. The modifications made to the gene gun allowed transfection of purkinje cells, see FIG. 6G where a transfected purkinje cell was detected at 225 μm, in elephant nose fish organotypic cerebellum slices at a depth greater than 200 μm using gas pressure at 75 psi. Transfection of purkinje cells was not possible using the BIO-RAD gun even with an optimum gas pressure of 175 psi. Thus, with the gun in accordance with the present invention, gold/DNA particles penetrate deeper into the organotypic slices so allowing transfection of purkinje cells.

Transfection of Dorsal Root Ganglions and Spinal Cord Explants

Rat dorsal root ganglions (DRG's) are less than 2 mm in diameter, tissue of this size cannot be transfected effectively using the BIO-RAD gene gun.

The DRG and spinal cord explants were cultured for 12 hrs before being ‘shot’ with EYFP CMV plasmid with gas pressure of 75 psi using the gun of FIG. 2, followed by a further two-five days in culture. Explants were placed onto membrane supports as described above (culture media comprised DMEM supplemented with 1% non-essential amino acids and with 10% FCS (foetal calf serum). Mammalian cells expressing EYFP were first detected after 24 hrs in culture. According to our observations the most intense EYFP-fluorescence in the mammalian explants was two days after transfection. DRG explants were fixed in 4% PFA and 300 μm sections cut using the tissue chopper. The DRG explants demonstrated the accuracy achieved with the gun of FIG. 2, see FIG. 6A obtained using a X40 objective lens, with a scale bar of 50 μm, and enlarged in FIG. 6B with a scale bar of 20 μm, obtained using X100 oil immersion objective lens. The spinal cord explants were cultured for up to five days following transfection; fixed in 4% PFA and 40 μm sections cut using a freezing microtome. The free-floating sections were counterstained using Hoechst 33342 (0.01%) for 5 min, see FIG. 6C obtained using X25 objective lens, scale bar 100 μm, mounted on slides and analysed by confocal and fluorescent microscopy. Explants of rat spinal cord demonstrated the increased penetration depth achieved with the modified design. We were able to observe transfected cells at a depth of 350 μm, see FIG. 6D where arrows point to positive EYFP expression cells in the white matter of the spinal cord, scale bar 100 μm, and FIG. 6E showing enlargement of the same section with scale bar of 20 μm and arrow indicating a positive EYFV expression cell. At this depth the majority of cells expressing EYFP were glial cells in the white matter of the spinal cord. With the BIO-RAD gene gun, no EYFP-expressing glial cell expression was observed in the spinal cord.

### SUMMARY

Our results demonstrate that successful transfection of neuronal tissue deep within organotypic slices and tissue explants is possible with a gene gun in accordance with the present invention and as shown in FIG. 2. The gene gun is also suitable for in-vivo gene delivery. An increased efficiency of transfection is achieved compared to conventional techniques, such as lipofection, (Holt et al., 1990), calcium phosphate precipitation (Werner et al. 1990), and electroporation (Oellig et al., 1990) which are often unsuccessful for neuronal tissue. The modifications allowed an increased accuracy and greater tissue penetration whilst using a reduced helium pressure (greater than 50%) which reduces tissue damage. The BIO-RAD gun provides sufficient transfection efficiencies when using established cell lines, however it is rather limited in its applications to tissue slices, explants and iii-vivo. This is because of the large spread of particles, low tissue penetration depths and relatively high gas pressures employed in the BIO-RAD gun.

By focussing the helium gas into a narrower beam within the accelerator channel as in the gun in accordance with the present invention and introducing a 70 μm nylon mesh directly over the barrel, the gas pressure required is reduced while still providing a greater particle penetration depth (over 250 μm) and preventing agglomeration of particles.

The BIO-RAD design using helium gas at the optimum pressure, resulted in a lower concentration of gold/DNA particles on the explants and thus, a significantly less efficient transfection (data not shown). We believe that the higher transfection efficiency achieved with the modified design was due to the reduced spread and increased density (particles per unit area). The reduced spread also resulted in an increased accuracy and depth penetration; permitting transfection of smaller and deeper tissues.

The advantages of a biolistic technique are speed and convenience. There is no labour-intensive vial preparation; other than purchasing a gene gun and a tissue chopper there are no requirements for special equipment. In biolistic experiments, cotransfection with two or more different DNA-plasmids can be achieved within hours. The viral technique requires time-consuming viral vector preparation and often compromises neuronal viability (Moriyoshi et al., 1996, Giger et al., 1997, Jareb et al., 1998). The hand-held gene gun is easy to use with little training required. However, fine-tuning of experimental parameters is necessary for each application. Optimisation of these include bombardment parameters such as the helium gas pressure, the PVP concentration, and the amount of DNA loaded per mg of microcarriers (a term know as DNA loading ration (DLR)).

The technique has had a tremendous impact on plant and microbial research. The gene gun in accordance with the present invention makes the technique applicable for mammalian in-vivo as well as in-vitro use. A potential in-vivo application would be molecular modification, by particle-mediated gene transfer of human melanoma cells. Genes such as those encoding interferon-gamma (IFN-gamma) or the B7-1 costimulatory molecule (CD80), (Albertini et al., 1996, Mahvi et al., 1997, Fensterle et al., 1999) could be introduced and present a promising strategy to stimulate antimelanoma T-cell immunity. The gene gun has been used for regression of established primary and metastatic murine tumours (Rakhmilevich et al., 1996, Kolesnikow et al., 1995). Gene therapy aims to introduce specific genes into a host to replace defective ones (replacement therapy) or to suppress expression of certain undesirable genes (anti sense therapy). The hand-held gene gun is a new gene transfer technology, which is developing rapidly and represents a powerful tool for medical research.

To date, the hand-held gene gun has been useful for “in-situ” transfection of cells in mammalian skin (Johnston & Tang et al., 1993), but has limited effectiveness for transfection of deeper tissues (Hui et al., 1994). The gene gun of FIG. 2 with redesigned barrel allows an increase in accuracy, combined with greater penetration, without increasing the propulsive pressure, this could be due to the reduction in the coanda effect and the conversion from turbulent helium flow to laminar flow.

### REFERENCES

Albertini M R, Emler C A, Schell K, Tans K J, King D M, and Sheedy M J. Dual expression of human leukocyte antigen molecules and the B7-1 costimulatory molecule (CD80) on human melanoma cells after particle-mediated gene transfer. Cancer Gene Ther 1996,3: 192-201.

Andree C, Swain W F, Page C P, Macklin M D, Slama J, Hatzis D, and Eriksson E. In vivo transfer and expression of a human epidermal growth factor gene accelerates wound repair. Proc. Natl. Acad. Sci 1994, 91: 12188-12192.

Biewenga J E, Destree O H, and Schrama L H. Plasmid-mediated gene transfer in neurons using the biolistics technique. Journal of Neuroscience Methods. 1997:71, 67-75.

Cheng L, Ziegelhoffer P R and Yang N S. In vivo promoter activity and transgene expression in mammalian somatic tissues evaluated by using particle bombardment. Proc. Natl. Acad. Sci 1993, 90: 4455-4459.

Dong Y, Skoultchi A I, and Pollard J W. Efficient DNA transfection of quiescent mammalian cells using poly-L-omithine. Nucleic Acids Research 1993, 21: 771-772.

Fensterle J, Grode L, Hess J, and Kaufmann S H. Effective DNA vaccination against listeriosis by prime/boost inoculation with the gene gun. J. Immunol 1999, 163(8): 4510-4518.

Giger R J, Ziegler U, Hermens W T, Kunz B, Kunz S, and Sonderegger P. Adenovirus-mediated gene transfer in neurons: construction and characterization of a vector for heterologous expression of the axonal cell adhesion molecule axonin-1. J. Neurosci Methods 1997, 71: 67-75.

Holt C E, Garlick N, and Comel E. Lipofection of cDNA's in the embryonic vertebrate central nervous system. Neuron 1990, 4: 203-214.

Hui K M, Sabapathy T K, Oei A A and Chia T F. Generation of allo-reactive cytotoxic T lymphocytes by particle bombardment-mediated gene transfer. J. Immunol Methods. 1994, 171: 147-155.

Imants R. Applications of the coanda effect. Scientific American. 1966, 214: 84-92.

Jareb M and Banker G. The polarized sorting of membrane proteins expressed in cultured hippocampal neurons using viral vectors. Neuron 1998, 20: 855-67.

Johnston, S A and Tang, D C. The use of microparticle injection to introduce genes into animal cells in vitro and in vivo in genetic engineering. (Setlow, J. K., ed.). Plenum Press, New York, pp 225-236 (1993).

Katz L C, Lo D C, and McAllister K. Neuronal transfection in brain slices using particle-mediated gene transfer. Neuron 1994, 13:1263-1268.

Klein T M, Wolf E D, Wu R and Sanford J C. High velocity microprojectiles for delivering nucleic acids into living cells. Nature 1987, 327: 70-73.

Klein T M, Fromm M, Weissinger A, Tomes D, Schaaf S, Sletten M, and Sanford J C. Transfer of foreign genes into intact maize cells with high-velocity microprojectiles. Proc. Natl. Acad. Sci. 1988, 85: 4305-4309.

Kolesnikov V A, Zelenina I A, Semenova M L, Shafei R, and Zelenin A V. The ballistic transfection of mammalian cells in vivo ontogene 1995, 6: 467-480.

Mahvi D M, Sheehy M J, and Yang N S. DNA cancer vaccines: a gene gun approach. Immunol Cell Biol 1997,(5): 456-460.

Moriyoshi K, Richards L J, Akazawa C, O'Leary D D, and Nakanishi S. Labelling neural cells using adenoviral gene transfer of membrane-targeted GFP. Neuron 1996, 16: 255-60.

Oellig C, and Seliger B. Gene transfer into brain tumour cell lines: reporter gene expression using various cellular and viral promoters. Neurosci Res 1990, 26: 390-396.

Rakhmilevich A L, Turner J, Ford M J, McCabe D, Sun W H, Sondel P M, Grota K, and Yang N S. Gene gun-mediated skin transfection with interleukin 12 gene results in regression of established primary and metastatic murine tumors. Proc. Natl. Acad. Sci. 1996, 13: 6291-6296.

Sanford J C, and Smith F D, Russell J A. Optimizing the biolistic process for different biological applications, Meth. Enzymol. 1993, 217: 483-509.

Sato H, Hattori S, Kawamoto S, Kudoh I, Hayashi A, Yamamoto I, Yoshinari M, Minami M, and Kanno H. In vivo gene gun-mediated DNA delivery into rodent brain tissue. Biochem Biophys Res Commun 2000,270:163-170.

Wellmann H, Kaltschmidt B, and Kaltschmidt C. Optimized protocol for biolistic transfection of brain slices and dissociated cultured neurons with a hand-held gene gun. Journal of Neuroscience Methods 1999, 92: 55-64.

Werner M, Madreperla S, Lieberman P, and Adler R. Expression of transfected genes by differentiated postmiotic neurons and photoreceptors in primary cell cultures. J.Neurosci Res. 1990, 25: 50-57.

Williams R S, Johnston S A, Riedy M, DeVit M J, McElligott S G, and Sanford J C. Introduction of foreign genes into tissues of living mice by DNA-coated microprojectiles. Proc. Natl. Acad.Sci. 1991, 88: 2726-2730.

Yang N S, Burkholder J, Roberts B, Martinell B, and McCabe D. In vivo and in vitro gene transfer to mammalian somatic cells by particle bombardment. Proc. Natl. Acad. Sci. 1990, 87: 9568-9572.

Yang N S, Sun W H, and McCabe D. Developing particle-mediated gene-transfer technology for research into gene therapy of cancer. Mol Med Today 1996, 11: 476-481.

1. A biolistic device for accelerating a carrier gas bearing particles, the device comprising an elongate body with a cylindrical channel extending therethrough, wherein a first end of the channel is adapted to receive gas for acceleration and a second end of the channel is adapted to direct gas to a target, thereby to produce a compact output beam of carrier gas from an output orifice.

2. A biolistic device according to claim 1, wherein the elongate body comprises an accelerating section and an output section, the channel extending through both the accelerating section and the output section, the output section having a plurality of apertures extending at an angle from an inner wall of the output section to an outer wall of the output section.

3. A biolistic device according to any of the preceding claims, wherein the apertures are inclined at an angle of 30°.

4. biolistic device according to any of the preceding claims, wherein a mesh is disposed over the output orifice so as to reduce agglomeration of particles in the output beam of carrier gas.

5. A biolistic device according to claim 4, wherein the mesh is secured over the output orifice by an annular member.

6. A biolistic device according to any of the preceding claims, wherein the cylinder is made of plastics material, with the output section being made either of plastics material or of metal.

7. A biolistic device according to any of claims 2 to 6, wherein the elongate body comprises a seperable accelerating section and output section.

8. A biolistic device according to claim 7, wherein the accelerating section is provided with a cylindrical recess, the recess engaging with the output section with a pressfit connection.

9. A biolistic device according to any of the preceding claims, wherein the elongate body is substantially T-shaped, with sealing means provided at the base of the T, and a connecting means provided on the neck of the T.

10. A biolistic device according to any of the preceding claims, wherein a guide rod is attached to set the distance from a target.

11. An elongate body adapted to retrofit to a BIO-RAD gene gun, the elongate body having cylindrical channel extending therethrough.

12. An elongate body according to claim 11, wherein the elongate body is substantially T-shaped, with sealing means provided at the base of the T, and a connecting means provided on the neck of the T.

13. An elongate body according to claim 11 or claim 12, wherein the body comprises an accelerating section and an output section, the channel extending through both the accelerating section and the output section, the output section having a plurality of apertures extending at an angle from an inner wall of the output section to an outer wall of the output section.

14. A biolistic device according to any of claims 1 to 10 when used for in-vivo transfection.
